SEQUENCE LISTING (28 sequences)

SEQ ID NO: 1 - DNA, 1361 bp, Artificial Sequence
  modified enhancer
caacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctagatccccaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctagatccccaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctagatccccaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctagatccgaaact

SEQ ID NO: 2 - DNA, 202 bp, Artificial Sequence
  enhancer fragment
agctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatc

SEQ ID NO: 3 - DNA, 129 bp, Artificial Sequence
  enhancer fragment
caacatggtggagcacgacactctcgtctactccaagaatatcaaagatacagtctcagaagaccagagggctattgagacttttcaacaaagggtaatatcgggaaacctcctcggattccattgccc

SEQ ID NO: 4 - DNA, 7 bp, Artificial Sequence
  enhancer fragment
agatccc

SEQ ID NO: 5 - DNA, 313 bp, Artificial Sequence
  promoter fragment
agctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactgaaccaatatttctcactctaagaaattaagagctttgtattcttcaatgagaggctaagacc

SEQ ID NO: 6 - DNA, 207 bp, Artificial Sequence
  enhancer fragment
gtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaa

SEQ ID NO: 7 - DNA, 250 bp, Artificial Sequence
  enhancer fragment
gagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacgatcgagaagacacggccat

SEQ ID NO: 8 - DNA, 360 bp, Artificial Sequence
  promoter  fragment
ttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatct

SEQ ID NO: 9 - DNA, 24 bp, Artificial Sequence
  oligonucleotide primer
agcggataacaatttcacacagga

SEQ ID NO: 10 - DNA, 20 bp, Artificial Sequence
  oligonucleotide primer
ttatttcttgagggcctcga

SEQ ID NO: 11 - DNA, 20 bp, Artificial Sequence
  oligonucleotide primer
cggcaatgtaccagctgata

SEQ ID NO: 12 - DNA, 19 bp, Artificial Sequence
  oligonucleotide primer
gatccccaacatggtggag

SEQ ID NO: 13 - DNA, 21 bp, Artificial Sequence
  oligonucleotide primer
cacatcaatccacttgctttg

SEQ ID NO: 14 - DNA, 20 bp, Artificial Sequence
  oligonucleotide primer
actacgatacgggagggctt

SEQ ID NO: 15 - DNA, 20 bp, Artificial Sequence
  oligonucleotide primer
ctggcgtaatagcgaagagg

SEQ ID NO: 16 - DNA, 20 bp, Artificial Sequence
  oligonucleotide primer
tgacatgctccaaattccaa

SEQ ID NO: 17 - DNA, 20 bp, Artificial Sequence
  oligonucleotide primer
cttggcattgggatcaaact

SEQ ID NO: 18 - DNA, 22 bp, Artificial Sequence
  oligonucleotide primer
tttctttcacagatccgagtca

SEQ ID NO: 19 - DNA, 20 bp, Artificial Sequence
  oligonucleotide primer
ttctccacactgcagattcg

SEQ ID NO: 20 - DNA, 20 bp, Artificial Sequence
  oligonucleotide primer
gaggattgcccaaaaccata

SEQ ID NO: 21 - DNA, 20 bp, Artificial Sequence
  oligonucleotide primer
ttttgggtgcaaaaacatca

SEQ ID NO: 22 - DNA, 20 bp, Artificial Sequence
  oligonucleotide primer
taatacgactcactataggg

SEQ ID NO: 23 - DNA, 22 bp, Artificial Sequence
  oligonucleotide primer
cgaggatatgaaatctcttgcc

SEQ ID NO: 24 - DNA, 21 bp, Artificial Sequence
  oligonucleotide primer
ggcaagagatttcatatcctc

SEQ ID NO: 25 - DNA, 20 bp, Artificial Sequence
  oligonucleotide primer
tcagcaaatgcagaggtttg

SEQ ID NO: 26 - DNA, 4436 bp, Artificial Sequence
  L23 sequence from plasmid rescue
gtcgtcagtaataatgttttcctggtgtatcgaacatcagagaaagaagttaaaattaaactgtatcaacacggtctacaaactttgcatttctcatgtgaaaggcgtcaaagaggggaagaagaagagaaagattaacgaaaagacatcaaacgttcaccttttgcagcaggtccattttcttcttaatgccagcagcaccatcctccttcaacaactgcttaatctgcatgaaaggccacaagttaaaagactagcaaatgaggtgcaactacattgctcggactctataaaaatgttgttgcccctgtgtcggatcctacttttggaggatctgacatgctccaaattccaacttaatcttcacggaagtcctattaccccctgaacgttttttttatatattaggtcacccttataggctaatgtgtccacaaacgtgaactcacctccgtgagggatgtaagaaccaaataaatgtgtacatctggagtccaaataagcttccaatgtgcattttatttggttcttacgcgcttcacggaggtgagttcacgcttgtggacacaccagcctataaggatgaacaaatattaaaaagaaaagtttccggggtaataggactcccgtaaagattttgcagttggaaatacggctaaagcttgagggtgcattcgaccattttctcaaggatattaggaacaataagggacttcaacattggcccaacctattatattatttcttttttatgactgagaagttggactgaagccacccttcaggtcattttccacttaaatactagactttgttcgttcacttggcattgggatcaaacttgtgaccgaaaagacacaagtccctttcaatctgggttaatttattttttctgtttctgtctgcccacttttttgaatattatctttttattaaactaaaaaaatatcaaataagtttggcaaacacaataggaaatattctagttttacatttgaggataacatcactttttattcggtaatatgacatgagatgagcttaaatggagaaatagcgtcagttatgatccatatagacaaacccaacttctctggactaagacgtagttattgtagtaaagatcacaacaggacctgggtagagagactgatccttcaacaactaatttgattaagttggagaaacttcaaaacttatgtctattcatcttaaattttagataagatctttaaccacactagaactcagcaaactgattctacttattcagcatggtcagagtaagtagctaaggcccttacatcaactttgattgtattctttttacctacaaactatagagtgtgaaagaactacattgagcacatagtatgcaaaatttctttcacagatccgagtcaaatattgcttattaagggattaaaaatcgactattttttaaatgagtcgaatattgacttagtaataattatttattactaacatatttaggattccaggagagattgttttcacctcattcctggacgtccttgtgtaaggagcaggttactcgggtagaacttagcatagcatctgacatatggactgatggtcagtccgtgtggaccattcagcccaatccatgtatataatgttgttcattatgctttacttctttttttagacatagggtcatcgatacaatcattttttctaggcagatgtatcaaactagaagaaaaggtgaatgtttactttttatatgtattttaaatacttgatcaaacatctatggtatagattctgaatgaaaacatgcctaccttaatcagttccttttggcaggcggaagatatcttttcctcgtcatcatatctttttctccacactgcagattcgtccattgcttctctactggcaagatcgaactgtctcctacaagaatccaagaagaatatacttagccaagcattaaatggcatcgaacatatgtttgattgccatctttagactcgtgctgggaatgtggaatgattaaaaaccttggtcctgaaaacaaagtttcgtatttatttgaatcactactcaagttggaagcggttttgcatcggcaacttagagaaaacagtgcaatgttttcataacattctagtagaaactagttctaatagtttgatatatgccaataaaaaccacctctatagtcaaaccccatgtcaagtcacctattaaagcatttacaatagagttcacatgattttcacctgctaaagaaaataaacattgggcctaactcaacttcaaaagctagtttatgcaatgaggattgcccaaaaccatataaagagactaattgaactatcccccatatatgtgggacttccaacactcccgcatgcctaggcctgcaattggagcatggacaacataatatgagggcaaacatcgggtaaaccaagaaccaggacgggtgcaaagacataatcttttttaggtacattacaatgctggatacaagtaaaggtgattttgcttactttttttttttgattttttttgcttcaaattaagattaatcaacaaccccacttttcccagcaaccaaacaaagtgaaattccatttctaatcaatgttttaacccatttctgagtgcaaagacatgatctttttgaggtacatacaaaagattaagcaagaacattaaaacaaaggttaattttcatgattgagtgttaagagtaatcaaaaattcccaactagtccttctaattcttgaactactcccaatttttcccaacaaaacgaataaagtgaagaaccatttccaattattttgctgatccatttttgggtgcaaaaacatcatcttttttaggtacattccaatcatgggcggagttagaaacccaattaccaatagtttttgctcaaatagctacaacaacttacctagtgtaatctcacgaatggggtttggggcccctaccttgggaggcttttgctcagattgtgtatttatgctaagaaattcaacatacattatgtttaaaactcaaatatttggtcgttctaaaatcatggctctctaattccaatgccggatacaagggtgactgactgcttctttttttccttctttttaatgttcttgcttaaaactaaaactaatcaacatccccaaaagaccccaactagtcctttctgtttcttgaaccacacccagttttcccaccacccaaacaaagtgaaaatccattaccttggtgcaaaaacatgacttttttgaggtacagtccaatacttggataaaagtaaaggtgtttgttcttttttcccaatgttatgcttaaagttaagattaatcaacatcctcaatcactccaaacattttcttgattcaaagacattatctttgtgatctacatacaagtaaaggtgattgctttttcaatgttcttgctcaaaatcataaaattaatcaacatccccaattagtcctccagtttttgaactacacccaattttcccaacaaaccaaacaaagtgaaaatccattcccaaccattttcttgtttcaaagacatgatcttttttacttttagttacatcccaagtaaagataattgatttttcaacatccccaatcagtcctccagtttcttagcctacacccatttccccccaccaaccaaacaaaagtgaaaatccatttcttggtgcaaaaacatgatctttttgaggtaaaataaacaagaaacaagaagaagagaggtacctgagtaattcagtttcattagatgagaaattgtagtcaatggtccaaagccttaagtaaacagctaaacccatcaccataacaagaccaagtcccaaaaatatttgctttcttcctcctcccatcttctccttctttctcccaaacctctgcatttgctgatttctaggtttctcaaaaaaaaaaaaaacttaactactgtctgtaatttgggattaatgatggtaattggtttaatttttgacttaaataagatattaaggtaatggtaattgattggtcaatttctttcgaaagaaaaatttcaaaaattaaattatgttatattaatttaatattttaaacagaaaatttagatattcaaaaacgatatgaaaagtattataaattgtaattttttgtatattataaaatattaatcaaacttcttatcatttaactttaaaaaagaaataatgacaatataaagcggaagagtgagtaattttctagttttatgcaacattttttttttagtttttactttattttataaaagatttttgttattttatataatgataaagaaaagattttttttttttaattctgatattaaaaagggcaagagatttcatatcctcgatattatttatacggtgtgaagtaatatattttaatttttttttatgaaa

SEQ ID NO: 27 - protein, 124 aa, Artificial Sequence
  ORF from plasmid rescue of L23
MFSWCIEHQRKKLKLNCINTVYKLCISHVKGVKEGKKKRKINEKTSNVHLLQQVHFLLNASSTILLQQLLNLHERPQVKRLANEVQLHCSDSIKMLLPLCRILLLEDLTCSKFQLNLHGSPITP

SEQ ID NO: 28 - protein, 85 aa, Artificial Sequence
  ORF from plasmid rescue of L23
MGLAVYLRLWTIDYNFSSNETELLRRQFDLASREAMDESAVWRKRYDDEEKISSACQKELIKFVGKKNTIKVDVRALATYSDHAE

(19) United States
(12) Patent Application Publication
(10) Pub. No.: US 2002/0157130 A1
(43) Pub. Date: Oct. 24, 2002

(54) Trait-associated gene identification method

(76) Inventors: Ry Wagner, Eugene, OR (US); Helena Mathews, Portland, OR (US); Xing Liang Liu, Eugene, OR (US); Wendy J. Waggoner, Tigard, OR (US)

Correspondence Address: JAN P. BRUNELLE, EXELIXIS, INC., 170 HARBOR WAY, P.O. BOX 511, SOUTH SAN FRANCISCO, CA, 94083-0511, US

(21) Appl. No.: 09/522,334

(22) Filed: Mar. 9, 2000

Related U.S. Application Data
(60) Provisional application No. 60/124,232, filed on Mar. 12, 1999.

(51) Int. Cl. C12N 15/82

(52) U.S. Cl. 800/278

(57) ABSTRACT

The present invention provides a method for identifying and characterizing genes, the modified expression of which is associated with a desired trait in a fruit-bearing plant. The method includes the steps of: transforming plant cells with a plant cell expression vector comprising an element which functions to enhance gene expression and has the ability to integrate into the plant genome in a manner effective to enhance expression of native plant genes, selecting transformed plant cells, regenerating transformed plant cells to yield mature plants, selecting plants having a desired trait, identifying, isolating and characterizing genes the transcription of which has been enhanced and confirming the contribution of the modified expression of each identified gene to the desired trait.

This application claims priority to U.S. Provisional application Ser. No. 60/124,232, expressly incorporated by reference herein.

### FIELD OF THE INVENTION

The present invention relates to methods of identifying genes the enhanced expression of which results in plants having desired traits.

### BACKGROUND OF THE INVENTION

The development of fruit-bearing plants having desired traits such as improved yield, disease resistance, improved fruit ripening characteristics, improved food quality and improved appearance has been the focus of plant breeders and agribusiness for many years.

Traditional methods used to develop improved varieties of plants include cross-pollinating and grafting onto root stock, which are slow and labor intensive. It is now possible to produce plants which have new and/or improved characteristics of agronomic and crop processing importance using recombinant DNA technology. In general, applications of recombinant DNA technology to plant improvement have been focused on (1) generation of random mutations by the treatment of plants or seeds with mutagens, e.g., EMS (ethyl metanesulfonate) diepoxyoctane, diepoxybutane or gamma rays, (2) selective mutation of specific plant genes (3) introduction of heterologous gene constructs coding for modified native genes or heterologous genes into plants, and/or (4) the modified expression of native plant genes using various genetic control elements.

Native plant genes may be knocked-out or their expression modified by the various techniques employed to develop new plant varieties. The modified expression of endogenous plant genes takes the form of either up- or down-regulation of plant genes. Such up- or down-regulation of plant genes has been accomplished by manipulating the regulatory elements controlling transcription and/or translation of plant genes or by the introduction of nucleic acid constructs (sense or antisense), which enhance or reduce the expression of a given gene by modifying the transcription of that gene and/or translation of the mRNA encoded by that gene.

Insertional mutagenic techniques have also been used to generate random modifications of native plant genes. For example, the T-DNA insertion technique, termed “T-DNA tagging” or “activation tagging” has been used to develop large numbers of transformed plant lines, e.g., in Arabidopsis (Christensen, S., et al., 9^{th} INTL. CONF. ON ARABIDOPSIS RES. June 24-28, 1998, p 165, Univ. Of Wis.), as well as in the legume, Medicago truncatula (Kardailsky, I, et al., 9^{th} INTL. CONF. ON ARABIDOPSIS RES. June 24-28, 1998, p. 187-188, Univ. Of Wis.). In this technique, seeds are transformed with the Ti plasmid from Agrobacterium tumifaciens which is inserted randomly into the plant genome. [See, e.g., Feldmann, K. A., Plant J. 1:71, 1991; Hayashi H. et al., Science 258 (5086):1350-3, 1992; Walden, R., et al., Plant Molecular Biology, 26:1521, 1994]. The isolation of the floral inducer FLOWERING LOCUS T (FT), which acts in parallel with the meristem-identity gene LEAFY (LFY) to induce flowering in Arabidopsis using activation tagging has recently been described (Kardailsky I et al., Science 286(5446): 1962-5, 1999).

Arabidopsis thaliana is routinely used as a model for plant improvement, e.g., for insertional mutagenesis in Brassica species, which have a silique type of fruit. However, Arabidopsis does not serve as a model for plants having a fleshy fruit. (Meissner et al., The Plant Journal 12(6) 1465-1472, 1997).

Insertional mutagenesis using transposons has also been described, where a nucleic acid sequence comprising a natural or introduced transposon is induced to move to new locations throughout the plant genome. (See, e.g., U.S. Pat. No. 4,732,856.)

To date, activation tagging has not been shown to be practical in fruit-bearing plants, e.g., plants having a fleshy fruit such as tomato. Transposon tagging on the other hand, has proven a promising approach for mutagenesis and gene tagging in tomato using the Ac/Ds transposable element family (Yoder, et al., Mol. Gen. Genet. 213:291-196, 1988). Insertional mutagenesis by transposon tagging has also been successfully employed to develop plants having modified characteristics in Arabidopsis thaliana using the Ac/Ds transposable element system (Van Sluys, et al., EMBO J., 6:3881, 1987).

Currently available insertional mutagenic techniques have the capability of yielding important information about gene function in plants, however, in many cases the methods are time consuming, expensive and/or do not provide the desired information regarding genes associated with improved plant characteristics. The utility of such techniques is dependent on the ability to transform and screen large numbers of transformation events and the ability to identify genes associated with traits of interest which result from such transformation events.

Optimal methods of plant transformation vary dependent upon the type of plant. For example, Agrobacterium -mediated transformation of Brassica species has been optimized using hypocotyl tissue. (See, e.g., U.S. Pat. Nos. 5,750,871 and 5,463,174.) In contrast, in soybean, the preferred method for Agrobacterium-mediated transformation requires removal of the hypocotyl tissue. (See, e.g., U.S. Pat. Nos. 5,824,877 and 5,569,834).

The enhancement of gene expression provides a means to develop plants with new characteristics. Accordingly, efforts are being undertaken by both industry and academia to develop a means to identify and screen large numbers of gene activation events in plants in order to identify genes associated with plant traits or characteristics of interest in plants and to develop modified plants having such traits.

### SUMMARY OF THE INVENTION

The invention provides a “trait-associated gene identification method” for use in identifying, isolating and characterizing genes associated with “output traits” of interest in fruit-bearing plants.

In the preferred method of the invention, fruit-bearing plants are transformed with heterologous nucleic acid constructs (expression vectors) comprising an element which functions to enhance gene expression and stably integrates into the plant genome. A portion of the vector sequence may be used to locate and thereby identify and characterize the region of the native plant genome in the vicinity of the enhancer element.

Vectors for use in the methods of the invention have properties including the ability to: (1) insert and stably integrate into the native plant genome, (2) enhance transcription of native plant genes within 5000 bp or more of the insertion site, and (3) modify the phenotype of the plant when integrated and expressed in a plant.

Preferred vectors for use in the trait-associated gene identification methods of the invention comprise the following components: a nucleic acid sequence which facilitates replication and selection in E. coli; an element which functions to enhance gene expression, e.g., a tandem duplicated CaMV 35S enhancer; a selectable marker-encoding nucleotide sequence operably linked to a promoter effective to express the selectable marker encoding sequence; a termination element for said selectable marker-encoding nucleotide sequence; and a mechanism for stable integration of enhancer sequences into the plant genome, e.g., a T-DNA sequence.

Exemplary vectors include pSKI15, pAG 3201, pAG 3202 and pAG 4201.

In the methods of the invention, fruit-bearing plants may be transformed by any method known to stably introduce a heterologous gene construct into plants.

Preferred plants are those with short generation times, for example, a dwarf plant, e.g., tomato.

The preferred method of introducing nucleic acid sequences into plant cells is to infect a plant cell, an explant, a meristem or a seed with Agrobacterium tumefaciens, comprising a modified Ti plasmid which has a T-DNA sequence which facilitates enhanced expression of native plant genes by acting on endogenous promoters and which lacks tumor-causing genes.

In a preferred embodiment of the invention, a hypocotyl or shoot tip transformation method which does not require the use of feeder cells or nurse cultures is employed to introduce Agrobacterium vectors into plant cells.

Transformed explant cells are screened for the ability to be cultured in selective media having a threshold concentration of selective agent that is toxic to non-transformed plant cells, followed by culture under regeneration conditions to produce regenerated plant shoots, and transfer to a selective rooting medium to provide a complete plantlet, which may be grown to yield a mature plant.

A fraction of mature plants in which the expression of native genes is enhanced will exhibit desired traits. The plants which exhibit such desired traits are selected and the plant genomic DNA flanking the insertion site of the activation tagging nucleic acid construct identified and characterized. In a preferred embodiment, the sequence is identified using plasmid rescue and the extended sequence cloned in a cosmid vector.

The contribution of the identified gene to the desired phenotype is verified by transforming plant cells with a separate Agrobacterium binary expression vector comprising the nucleic acid sequences in the vicinity of the inserted enhancer which are associated with a trait of interest, and transforming plants with each separate binary expression vector.

Such transgenic plant cells are then screened for the ability to be cultured in selective media, cultured under regeneration conditions to produce regenerated plant shoots, and allowed to develop into mature plants, which are screened for desired traits.

In some cases, modified expression of a gene will result in a biochemical modification of the plant and/or fruit, such as a change in the level of vitamins, a change in the level of minerals, or elements, a change in the level of amino acids, a change in the level of carbohydrates, a change in the level of lipids, a change in the level of nitrogenous bases, a change in the level of isoprenoids, a change in the level of phenylpropanoids and a change in the level of alkaloids.

In other cases, modified expression of a gene will result in a trait such as increased resistance to fungal, bacterial or viral pathogens, increased resistance to insects, modified flower size, modified flower number, modified flower pigmentation and shape, modified leaf number, modified leaf pigmentation and shape, modified seed number, a modified pattern or distribution of leaves and flowers, modified stem length between nodes, modified root mass or root development characteristics, and increased drought, salt and antibiotic tolerance.

These and other objects and features of the invention will become more fully apparent when the following detailed description is read in conjunction with the accompanying figures and examples.

### BRIEF DESCRIPTION OF THE FIGURES

FIG. 1 is a schematic representation of the exemplary pSKI15 vector.

FIG. 2 is a schematic depiction of the steps involved in the overexpression of a gene by the activation tagging technique.

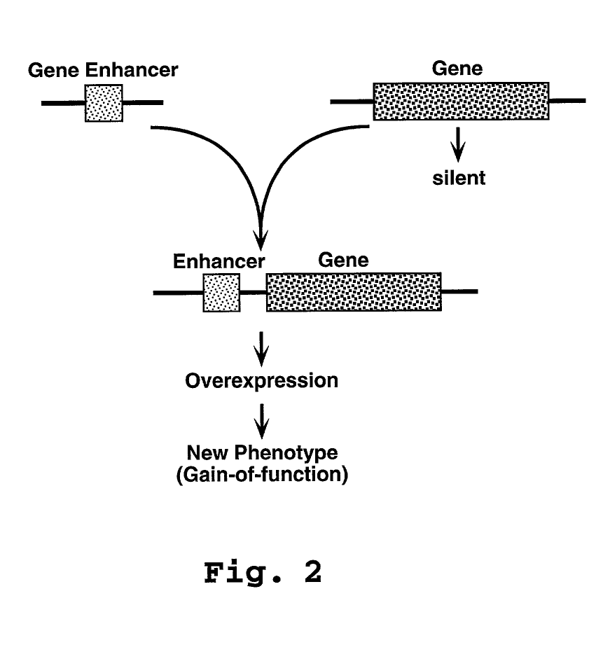

FIG. 3 is a schematic depiction of the steps involved in identification of a tagged gene related to a trait of interest, characterization of the identified gene and reintroduction of the identified gene into target crops.

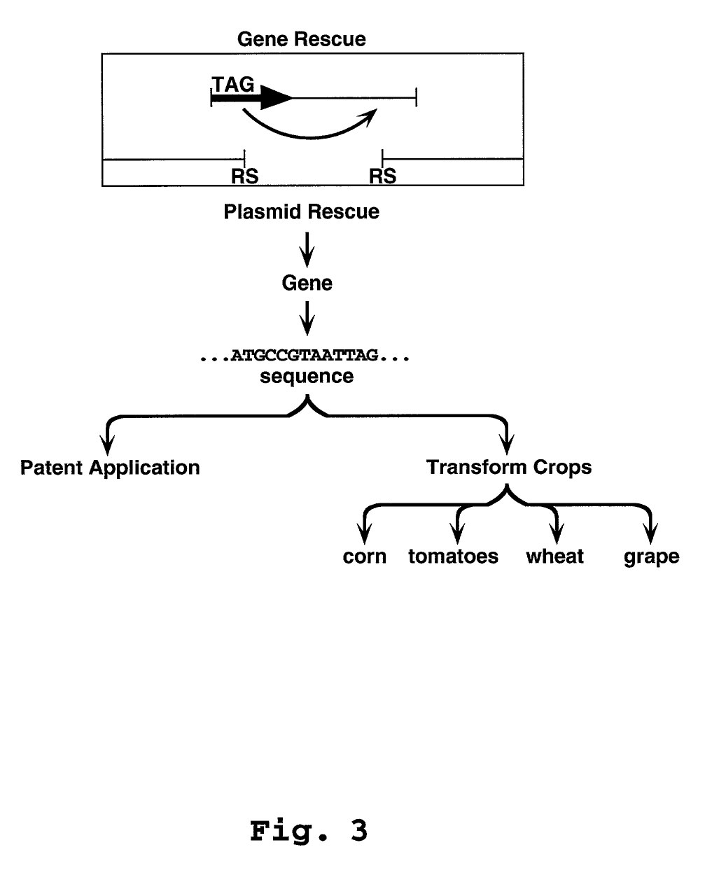

FIG. 4 is a schematic depiction of a 4X CaMV 35S enhancer acting in both directions on two genes at the same time and the results of screening 25,000 plants transformed with a genetic construct containing the 4X CaMV 35S enhancer.

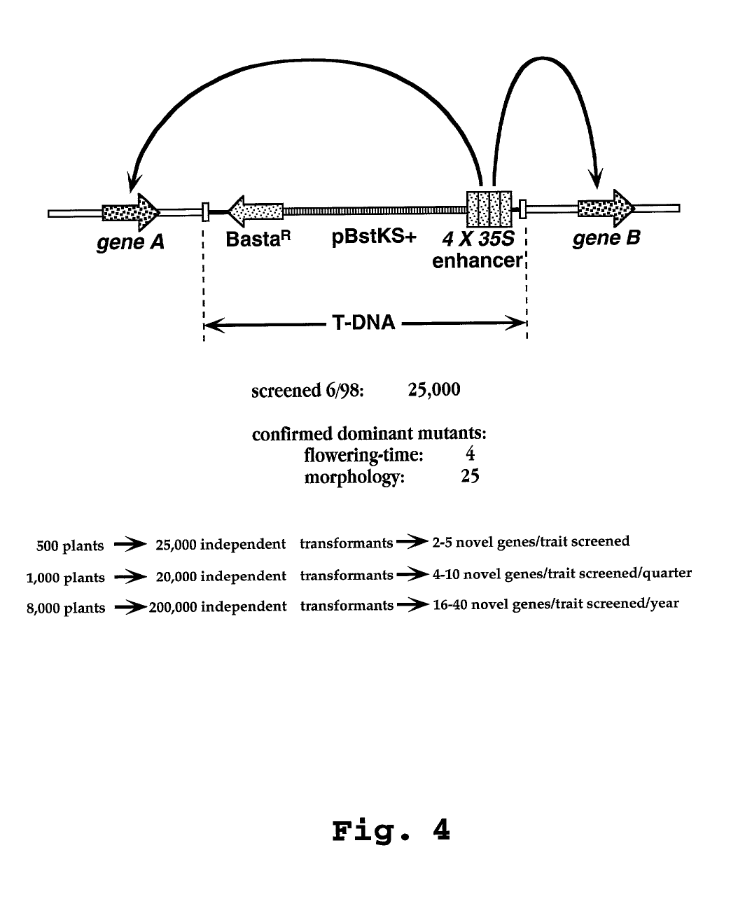

FIG. 5 depicts the 4X CaMV 35S enhancer sequence for use in the ACTTAG vector (SEQ ID NO: 1), including 4 Alul-EvoRV fragments in tandem (each 202 bp in length, SEQ ID NO: 2), an additional 129 bp of CaMV sequence (SEQ ID NO: 3), shown in italics and associated with each tandem Alu1-EcoRV repeat, and an additional 7 bp sequence (SEQ ID NO: 4), that is repeated and shown as underlined, wherein the restriction sites for Alul (AGCT) and EvoRV (GATATC) are shown in bold type.

FIG. 6 is a schematic representation of the exemplary pAG3202 binary plasmid, which has the pSKI backbone with a 4X 35S enhancer and the nptII selectable marker under the control of an RE4 promoter.

FIG. 7A depicts a photograph of the flower from an exemplary activation tagged Micro-Tom mutant, designated “L23” relative to a flower from a wild type Micro-Tom plant.

FIG. 7B depicts a photograph of an exemplary activation tagged Micro-Tom mutant plant, designated “L23” relative to a wild type Micro-Tom plant.

FIGS. 8A and 8B are schematic representations of two different plasmids with a 3.7 kb and 4.5 kb genomic insertion fragment derived by plasmid rescue using Xho I (9A) and Hind III (9B) digested L23 genomic DNA, respectively.

FIGS. 9A and 9B depict the 4437 bp DNA sequence obtained by plasmid rescue in L23.

FIG. 10A depicts the predicted amino acid sequence for a polypeptide of 124 amino acids based on the 4437 bp DNA sequence obtained by plasmid rescue from the Micro-Tom mutant L23.

FIG. 10B depicts the predicted amino acid sequence for a polypeptide of 85 amino acids based on the 4437 bp DNA sequence obtained by plasmid rescue from the Micro-Tom mutant L23.

### DETAILED DESCRIPTION OF THE INVENTION

I. Definitions

Unless otherwise indicated, all terms used herein have the same meaning as they would to one skilled in the art of the present invention. Practitioners are particularly directed to Sambrook et al. (1989) Molecular Cloning: A Laboratory Manual (Second Edition), Cold Spring Harbor Press, Plainview, N.Y. and Ausubel F. M. et al. (1993) Current Protocols in Molecular Biology, John Wiley & Sons, New York, N.Y., for definitions and terms of the art. It is to be understood that this invention is not limited to the particular methodology, protocols, and reagents described, as these may vary.

As used herein, the term “transgenic plants” refers to plants that have incorporated exogenous nucleic acid sequences, i.e., nucleic acid sequences which are not present in the native (“untransformed”) plant or plant cell.

As used herein, the term “activation tagging” refers to a process by which a vector having a nucleic acid control sequence, e.g. an enhancer, is inserted into a plant genome. The “tag” is a region of the nucleic acid sequence derived from the vector, which may be used to locate (“tag”), and thereby identify the point of insertion in the plant genome.

As used herein, the term “T-DNA sequence” refers to a sequence derived from the Ti plasmid of Agrobacterium tumifaciens containing the nucleic acid sequences which are transferred to a plant cell host during infection by Agrobacterium.

As used herein, the terms “enhancer” and “element which functions to enhance gene expression” may be used interchangeably and refer to any sequence which activates transcription of plant DNA from a nearby promoter. In the activation tagging methods of the invention, enhancers generally act to effect transcription of genes within 1000 to about 5000 or more bp of the insertion site.

The term “vicinity”, as used herein, regarding the relative location of a plant transcription initiation region and an enhancer sequence generally means the sequences are within about 5000 bp of one another, however, in some cases an enhancer may act at a distance of greater than 5000 bp.

As used herein, the term “selectable marker-encoding nucleotide sequence” refers to a nucleotide sequence which is capable of expression in plant cells and where expression of the selectable marker confers to plant cells containing the expressed gene the ability to grow in the presence of a selective agent.

As used herein, the term “Bar gene” refers to a nucleotide sequence encoding a phosphinothricin acetyltransferase enzyme which upon expression confers resistance to the herbicide glufosinate-ammonium (“Basta”).

As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

As used herein, the term “operably linked” relative to a recombinant DNA construct or vector means nucleotide components of the recombinant DNA construct or vector that are directly linked to one another for operative control of a selected coding sequence.

As used herein, the term “border sequence” refers to the nucleic acid sequence which corresponds to the left and right edges (“borders”) of a T-DNA sequence.

As used herein, the term “plasmid” refers to a circular double-stranded (ds) DNA construct used as a cloning vector, and which forms an extrachromosomal self-replicating genetic element in many bacteria and some eukaryotes.

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

A “heterologous” nucleic acid construct or sequence has a portion of the sequence which is not native to the plant cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like.

As used herein, the term “gene” means the segment of DNA involved in producing a polypeptide chain, which may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “sequence identity” means nucleic acid or amino acid sequence identity in two or more aligned sequences, aligned using a sequence alignment program. Sequence searches are preferably carried out using the BLASTN program when evaluating the of a given nucleic acid sequence relative to nucleic acid sequences in the GenBank DNA Sequences and other public databases. The BLASTX program is preferred for searching nucleic acid sequences which have been translated in all reading frames against amino acid sequences in the GenBank Protein Sequences and other public databases databases. Both BLASTN and BLASTX are run using default parameters of an open gap penalty of 11.0, and an extended gap penalty of 1.0, and utilize the BLOSUM-62 matrix. [See, Altschul, et al., Nucl. Acids Res. 25(17) 3389-3402 (1997).]

A preferred alignment of selected sequences in order to determine “% identity” between two or more sequences, is performed using the CLUSTAL-W program in MacVector, operated with default parameters, including an open gap penalty of 10.0, an extended gap penalty of 0.1, and a BLOSUM 30 similarity matrix.

A nucleic acid sequence is considered to be “selectively hybridizable” to a reference nucleic acid sequence if the two sequences specifically hybridize to one another under high stringency hybridization and wash conditions. Such conditions are recited in Ausubel FM et al. (1993) CURRENT PROTOCOLS IN MOLECULAR BIOLOGY, Suppl 21, John Wiley & Sons, New York, N.Y., expressly incorporated by reference herein.

As used herein, the term “expression” refers to the process by which a polypeptide is produced based on the nucleic acid sequence of a gene. The process includes both transcription and translation.

As used herein, the terms “transformed”, “stably transformed” or “transgenic” with reference to a plant cell means the plant cell has a non-native (heterologous) nucleic acid sequence integrated into its genome which is maintained through two or more generations.

Generally, a “variant” polynucleotide sequence encodes a “variant” amino acid sequence which is altered by one or more amino acids from the reference polypeptide sequence. The variant polynucleotide sequence may encode a variant amino acid sequence having “conservative” or “non-conservative” substitutions. Variant polynucleotides may also encode variant amino acid sequences having amino acid insertions or deletions, or both.

As used herein, the term “mutant” with reference to a polynucleotide sequence or gene differs from the corresponding wild type polynucleotide sequence or gene either in terms of sequence or expression, where the difference contributes to a modified plant phenotype or trait. Relative to a plant or plant line, the term “mutant” refers to a plant or plant line which has a modified plant phenotype or trait, where the modified phenotype or trait is associated with the modified expression of a wild type polynucleotide sequence or gene.

As used herein, a “plant cell” refers to any cell derived from a plant, including undifferentiated tissue (e.g., callus) as well as plant seeds, pollen, progagules and embryos.

As used herein, the term “mature plant” refers to a fully differentiated plant.

As used herein, the terms “native” and “wild-type” relative to a given plant trait or phenotype refers to the form in which that trait or phenotype is found in the same variety of plant in nature.

As used herein, the term “modified” regarding a plant trait, refers to a change in the phenotype of a transgenic plant relative to a non-transgenic plant, as it is found in nature.

As used herein, the term “phenotype” may be used interchangeably with the terms “trait” and “output trait”. The terms refer to a plant characteristic which is readily observable or easily evaluated and results from the interaction of the genetic make-up of the plant with the environment in which it develops. Such a phenotype includes chemical changes in the plant make-up resulting from enhanced gene expression which may or may not result in morphological changes in the plant, but which may be easily evaluated using analytical techniques known to those of skill in the art.

As used herein, the term “fruit texture” reflects the amount of soluble solids, total solids and cell wall components.

II. METHODS OF THE INVENTION

A. Enhancement of Gene Expression

The invention provides a trait-associated gene identification method based on the concept of “activation tagging”. Activation tagging is a process by which heterologous nucleic acid constructs comprising a nucleic acid control sequence, e.g. an enhancer, are inserted into a plant genome. Enhancers sequences can act to enhance transcription of a single gene or may enhance transcription of two or more genes at the same time. The enhancer sequence may insert within a native plant gene, “knocking out” that gene, within an intron of the gene, or between genes.

The “tag” is a region of the heterologous nucleic acid construct (i.e. the vector) which may be used to locate and thereby identify and characterize an introduced nucleic acid sequence that has been integrated in the plant genome. Such activation tagging nucleic acid constructs may be stably introduced into a plant genome in order to enhance expression (activate) native (endogenous) plant genes. (See, e.g., Walden R, et al., Plant Mol Biol 26(5),1521-8, 1994.)

Generally, vectors useful in the trait-associated gene identification method of the invention contain regions of the Ti plasmid of Agrobacterium tumifaciens which inserts preferentially into potentially transcribed regions of the plant genome. The vectors further contain transcriptional enhancer sequences which can activate gene expression at sites distant from the insertion point.

Appropriate vectors for use in the trait-associated gene identification methods of the invention are further described, below.

A fraction of the plants in which the expression of native genes is enhanced will exhibit desired traits. The plants which exhibit such desired traits are selected and the plant genomic DNA flanking the insertion site of the enhancer sequence of the activation tagging nucleic acid construct identified and characterized. Techniques routinely employed by those of skill in the art for identification and isolation of genes of interest are plasmid rescue [Behringer, F. J and Medford, J. I., Plant Mol. Biol. Reporter 10: 190-198 (1992)], and genome walking (e.g., GenomeWalker™ from Clontech, Palo Alto, Calif.).

Using the “tag”, the gene associated with a given desired trait may be cloned, for example using plasmid rescue to retrieve sequences of from about 100 to 3000 bp on either side of (flanking) the enhancer insertion site.

In some cases, inverse PCR may be used to isolate DNA adjacent known sequence in genomic DNA, by use of oligonucleotide primers complementary to one end of a known sequence that prime in opposite directions, and have a particular restriction enzyme site between them, e.g., the left or right border Ti sequences. In the method, chromosomal DNA is digested with a restriction endonuclease and ligated into a circularized DNA molecule. The resulting population of ligated molecules is comprised of a complex mixture of chromosomal DNA and chromosomal-vector DNA hybrids. The plasmid derived region of the hybrid molecules provides the downstream priming site for PCR amplification. The upstream primer may be specific for the vector, or a gene-specific primer. [See, e.g., Novak, J. and Novak, L., Promega Notes Magazine Number 61:27, 1997]

In an exemplary application of the methods of the invention, following introduction of an “activation-tagging” vector into plant cells and identification of plants having desired traits, sequences of from about 100 to 3000 bp flanking the enhancer insertion site are recovered by plasmid rescue. The rescued sequences are used to pull out longer native plant DNA sequences of from about 20 kb on each side of the enhancer insertion site and to construct cosmid clones containing from about 20 to 40 kb of the native plant DNA. The sequences in the cosmid clones are screened for open reading frames, and used to probe Northern blots of genomic DNA derived from a particular plant, e.g., tomato. Genes having altered expression in transformed plants relative to plants which have not been transformed are identified in this manner. (See, e.g., METHODS IN PLANT MOLECULAR BIOLOGY AND BIOTECHNOLOGY, Glick and Thompson Eds., CRC Press, 1993, pages 67-73 and 89-106). Methods for construction of cosmid clones are provided in chapter 3 of Maniatis, et al., MOLECULAR CLONING: A LABORATORY MANUAL, 2d Edition (1989), which is expressly incorporated by reference, herein.

Once a gene is identified, the nucleic acid sequence, including control and/or regulatory regions is isolated, cloned and characterized.

Northern blotting and RT-PCR (reverse transcriptase polymerase chain reaction) are used to confirm expression of genes associated with a desired trait.

The nucleic acid sequence of the identified gene, including control and/or regulatory regions may then be cloned and reintroduced into plants in a separate heterologous nucleic acid construct, e.g., a standard Agrobacterium binary vector, in order to enhance the expression of each identified gene and to independently confirm the contribution of each gene to the desired phenotype (trait). Again, plants having desired traits are selected and the genes associated with those traits used to develop improved plants having desired properties.

In some cases, once a gene associated with a desired trait has been isolated, characterized (i.e., sequenced), and its function confirmed, the sequence of the gene may be modified, for use in development of transgenic plants having desired phenotypes.

It will be appreciated that in most cases when a modified phenotype results from the enhanced expression of a tagged gene, the phenotype is dominant.

In some cases, the enhanced expression of a given native plant gene may result in decreased expression or inactivation of another native plant gene, which affects a desired trait.

Random expression of native genes may also be achieved by introduction of a nucleic acid construct comprising a transposon into the genome of interest. Exemplary transposons such as Ac, Ds, Mu or Spm are elements which can insert themselves into genes and cause unstable mutations. The mutations are unstable due to subsequent excision of the transposon from the mutant locus during plant or seed development. (See, e.g., Doring, H. P. and Starlinger (1986), Ann. Rev. Genet. 20:175-200; Federoff, N. (1989), “Maize Transposable Elements” in Mobile DNA. Wowe, M. M. and Berg, D. E., eds., Amer. Soc. Microbiol., Wash., D.C., pp. 377-411.) An exemplary transposon-tagging strategy used to identify a semi-dominant mutation affecting plant height, hypocotyl elongation, and fertility has been described. [See, Wilson K. et al., Plant Cell 8(4):659-71, 1996.]

Vectors containing transposons, particularly Ac, may be introduced in order to inactivate (or activate) and thereby “tag” the gene controlling a particular trait. Once tagged, the gene associated with the trait may be cloned, e.g., using the transposon sequence as a PCR primer together with PCR gene cloning techniques. Once identified, the entire gene(s) for the particular trait, including control or regulatory regions where desired, may be isolated, cloned and manipulated as desired prior to reintroduction into plants. Accordingly, transposable elements such as Ac, Ds, Mu or Spm may be incorporated into an activation tagging nucleic acid construct for use in the methods of the invention, in order to move an enhancer around the plant genome. Transposon-containing activation tagging nucleic acid constructs may or may not contain a selectable marker-encoding sequence.

An enhancer trapping and a gene trapping system, based on the AciDs maize transposable elements, has been transferred into tomato, and found to be active. (See, e.g., Yoder, et al., Mol. Gen. Genet. 213:291-296, 1988.) In addition, methods for generating unlinked and stabilized transposition of Ds, and for selection of excision and reinsertion, where linked transposition events are most often recovered, have been described (See, e.g., Sundaresan, Trends Plant Sci. 1: 184-190, 1996; Meissner et al., The Plant Journal 12(6) 1465-1472, 1997).

In one preferred embodiment of the methods described herein, the activation tagging vector is modified in a manner which allows for conditional disruption of the enhancer.

B. Plant Transformation

The method for introduction of vectors which effect enhanced expression of endogenous genes in plant cells is an important aspect of the invention. It is preferred that the vector sequences be stably integrated into the host genome.

Exemplary methods for transformation of plant cells in the trait-associated gene identification methods of the invention are Agrobacterium-mediated transformation, electroporation, microinjection, and microprojectile bombardment.

In the preferred embodiment, plant cells are transformed by infection with Agrobacterium tumifaciens. However, as will be appreciated, the optimal transformation method and tissue for transformation will vary depending upon the type of plant being transformed.

c. Transformation with Agrobacterium Vectors

The preferred method of introducing nucleic acid sequences into plant cells is to infect a plant cell, an explant, a meristem or a seed with Agrobacterium tumefaciens, a ubiquitous soil bacterium that infects a wide range of plants. Agrobacterium is capable of transferring a heterologous DNA sequence into infected plants, by way of the T-DNA from its tumor-inducing Ti plasmids. By removing the tumor-causing genes so that they no longer interfere, modified Ti plasmids are used as vectors for the transfer of selected nucleic acid sequences into plant cells. These Ti plasmids contain short directly repeated sequences which flank the T-DNA (termed left and right border sequences), and play a key role in the T-DNA integration. Upon infection by Agrobacterium tumefaciens, a heterologous DNA sequence is stably integrated into the plant genome in one or more locations.

In general, a selected nucleic acid sequence is inserted into an appropriate restriction endonuclease site(s) in the vector. Standard methods for cutting, ligating and E. coli transformation, known to those of skill in the art, are used in constructing vectors for use in the present invention.

Binary Ti-based vector systems are used to transfer and confirm the association enhanced expression of a given gene with the modified trait or phenotype of the plant. Appropriate vectors for this aspect of the invention include plasmids containing at least one T-DNA border sequence (left, right or both), restriction endonuclease sites for the addition of one or more heterologous nucleic acid sequences [adjacent flanking T-DNA border sequence(s)], a heterologous nucleic acid sequence (i.e., the coding sequence of identified and isolated genes), operably linked to appropriate regulatory sequences and to the directional T-DNA border sequences, a selectable marker which is functional in plant cells, a heterologous Ti-plasmid promoter, an E. coli origin of replication.

The Agrobacterium binary plant transformation vector is introduced into a disarmed strain of A. tumefaciens by electroporation (Nagel, R., et al., FEMS Microbiol. Lett. 67:325, 1990), followed by co-cultivation with tomato plant cells, to transfer the chimeric genes into plant cells.

In general, co-cultivation is carried out for two or three days in the absence of feeder cells or a nurse culture, as further described in Example 1.

Standard Agrobacterium binary vectors are known to those of skill in the art and many are commercially available, an example of which is pBI121 (Clontech Laboratories, Palo Alto, CA).

III. Vectors For Enhanced Expression Of Native Plant Genes

Preferred vectors suitable for use in the trait-associated gene identification methods of the invention comprise the following components: a nucleic acid sequence which facilitates replication and selection in E. coli; an element which functions to enhance gene expression, e.g., tandem duplicated CaMV 35S enhancer; a selectable marker-encoding nucleotide sequence operably linked to a promoter effective to express the selectable marker encoding sequence; a termination element for the selectable marker-encoding nucleotide sequence; and a mechanism for stable integration of enhancer sequences into the plant genome, e.g., a T-DNA sequence.

In some cases, a transposable element may be used to effect stable integration of enhancer sequences into the plant genome.

Nucleic acid sequences which facilitate replication and selection in E. coli, are well known to those of skill in the art. An exemplary E. coli sequence is the pBstKS + segment of the Bluescript™ KS+ plasmid sold by Stratagene.

Preferred vectors for use in the methods of the invention comprise an enhancer sequence having regions of the sequence which resemble that of a native enhancer. The enhancer domain comprises at least the same number of the repeats (repetitive nucleotide units) as a native enhancer and need not have more than the minimum number of repeats necessary for expression. In one embodiment, the vector has at least one natural enhancer sequence. In a preferred embodiment, the enhancer domain has two or more and generally four repetitive units of a natural enhancer sequence, in tandem in either orientation.

The enhancer domain is cis-acting and preferably located within about 5000 bp of the transcription initiation domain which is enhanced. It will be appreciated that in some cases, the enhancer domain will act on a transcription initiation domain which is more than 5000 bp away from the location of the enhancer sequence. The enhancer may be in forward or reverse orientation, with respect to the transcription initiation domain (promoter) can be located upstream or downstream relative to the promoter it enhances, generally upstream. In some cases, the enhancer is integrated within an intron.

The enhancer domain and promoter may be from the same or different species. However, the enhancer sequence necessarily comes from sources which function efficiently in plants. Usually the enhancer will be of viral or (higher) eukaryotic origin.

A promoter sequence may function as an enhancer. For example, a 68 bp element of the beta-phaseolin promoter has been demonstrated to function as a seed-specific enhancer (van der Geest A. H. and Hall T. C., Plant Mol Biol 32(4):579-88, 1996).

One preferred enhancer domain is from a virus (e.g., the CaMV 35S enhancer), and can enhance transcription from a transcription initiation region of a structural gene in a plant. An exemplary sequence for CaMV may be found in GenBank at Accession number X02606. The sequence delineates the 5′ region of the gene, which comprises the enhancer and promoter sequences.

In one embodiment, a vector for use in the methods of the invention, has an enhancer domain with at least one natural CaMV 35S enhancer sequence which is greater than 100 bp in length, preferably 200 bp to about 800 or 850 bp in length, derived from the native CaMV 35S genome.

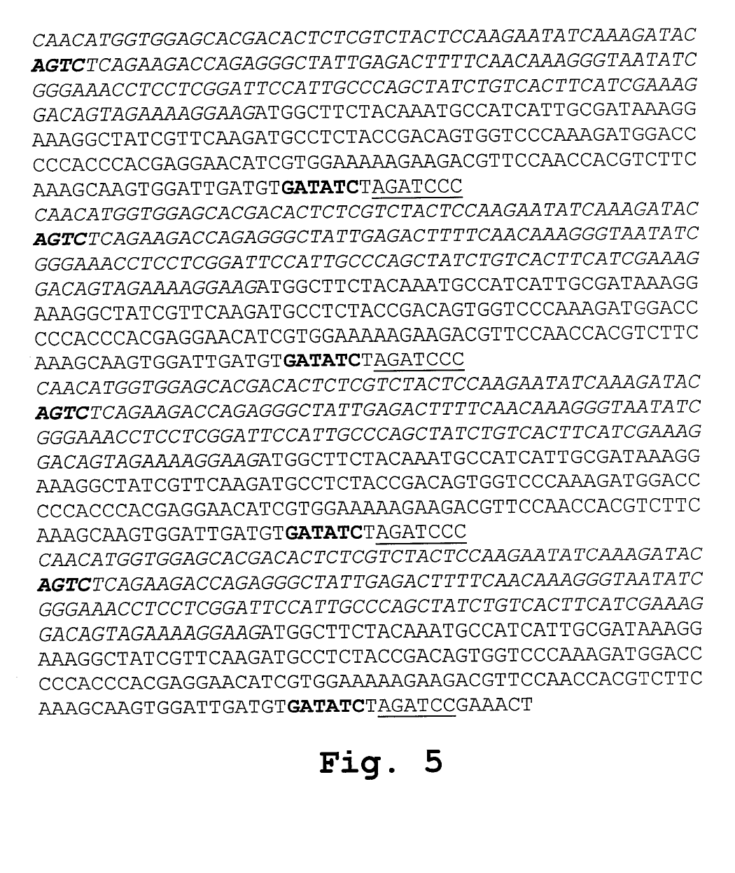

FIG. 5 depicts one preferred 4X CaMV 35S enhancer sequence (SEQ ID NO: 1), for use in the methods and compositions of the invention, including 4 Alu1-EvoRV fragments in tandem (each 202 bp in length, SEQ ID NO: 2), 129 bp of CaMV sequence (SEQ ID NO: 3), associated with each tandem Alu1-EcoRV repeat, and an additional 7 bp repeated sequence (SEQ ID NO: 4), which does not appear in the 35S enhancer region of the native CaMV genome.

Additional exemplary sequences which may function as enhancers include a sequence from Figwort Mosaic Virus (FMV, Maiti et al., Transgenic Res. 6: 142-156, 1997) Maiti et al., 1997, describes an FMV sequence with strong promoter activity, which corresponds to positions 6691 to 7003 of the complete FMV genome sequence found at GenBank Accession No. X06166 (SEQ ID NO: 5). An enhancer region is found at nucleotides 6678 to 6885 of the same sequence (SEQ ID NO: 6).

Another exemplary enhancer sequence is derived from peanut chlorotic streak caulimovirus (PClSV). The promoter for the full-length transcript (FLτ) of PClSV is described in U.S. Pat. No. 5,850,019 and in Maiti and Shepherd, Biochem. Biophys. Res. Commun. 244: 440, 1998, and corresponds to positions 5852 to 6101 of the complete genome sequence of PClSV (found at GenBank Accession No. U13988). The enhancer region is from 5852 to 6029 of the same sequence and has the sequence presented as SEQ ID NO: 7.

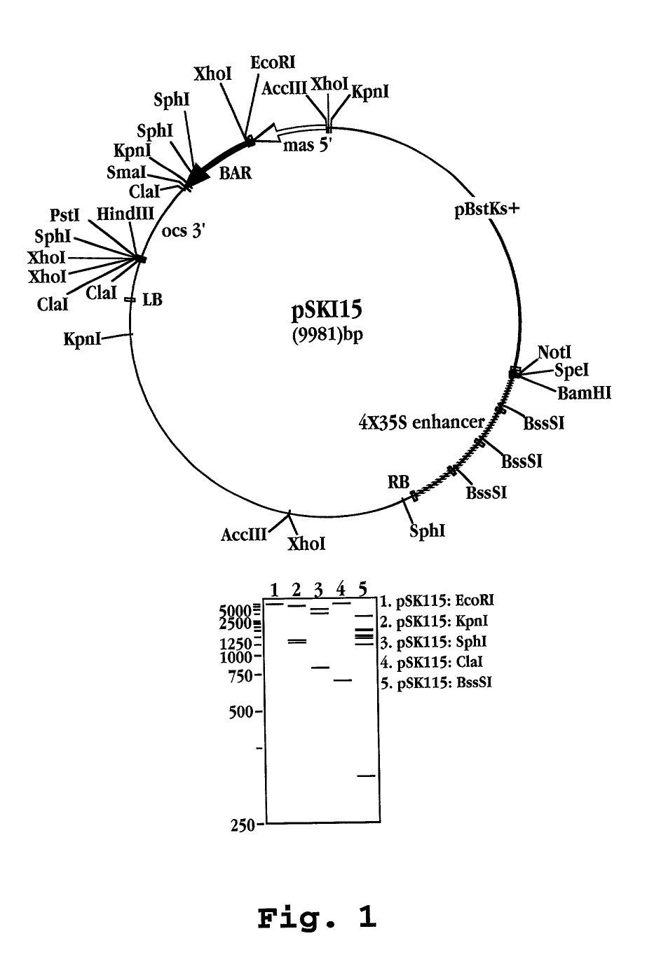

A further exemplary enhancer sequence is derived from mirabilis mosaic virus (MMV), a double-stranded DNA plant pararetrovirus belonging to the caulimovirus family. The complete genome sequence of MMV is unpublished. The sequence of the characterized MMV promoter fragment has been described by Dey and Maiti, Plant Mol. Biol. 40: 771, 1999, (FIG. 1). The fragment with the highest promoter activity extends from nucleotides −297 to +63 of the published sequence, and has the sequence presented as SEQ ID NO: 8. Within the promoter fragment, an enhancer region was identified which includes the sequence that extends from nucleotide −297 to −38 relative to the transcription start site of the published sequence (nucleotides 1-260 of SEQ ID NO: 8).

Marker genes which facilitate selection of transformants may encode either a selectable or screenable marker for use in plant cells, depending on whether the marker confers a trait which one can select for by chemical means, i.e., through the use of a selective agent (e.g., an herbicide, antibiotic, or the like), or whether it is simply a trait that one can identify through observation or testing. Numerous suitable marker genes known in the art may be employed in practicing the invention.

Exemplary selectable markers for use in the vectors of the invention include but are not limited to antibiotic resistance genes, such as, kanamycin (nptII), G418, bleomycin, hygromycin, chloramphenicol, ampicillin, tetracycline, or the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance; or a methotrexate resistant DHFR gene.

In one embodiment, the methods of the invention are carried out using a vector carrying the kanamycin resistance gene. In another embodiment, the methods of the invention are carried out using a vector which includes the bar gene from Streptomyces, which encodes phosphinothricin acetyl transferase (PAT, Akama, et al., 1995) that inactivates the active ingredient in the herbicide bialaphos, phosphinothricin (PPT). PPT inhibits glutamine synthetase, causing rapid accumulation of ammonia and cell death. Transgenic plants containing this gene exhibit tolerance to the herbicide, “BASTA”. This gene can also be used as a selectable marker gene, since explants carrying the bar gene are capable of growing on selective media containing phosphinothricin (PPT), which is an active component of bialaphos.

In further embodiments, the methods of the invention are carried out using a vector which includes an herbicide resistance gene, conferring resistance to glyphosate-containing herbicides. Glyphosate refers to N-phosphonomethyl glycine, in either its acidic or anionic forms. Herbicides containing this active ingredient include “ROUNDUP” and “GLEAN”. Exemplary genes for imparting glyphosate resistance include an EPSP synthase gene (5-enolpyruvyl-3-phosphosshikimate synthase) (Delanney, et al., 1995; Tinius, et al., 1995), or an acetolactate synthase gene (Yao, et al., 1995).

The particular marker gene employed will be one which allows for selection of transformed cells as compared to cells lacking the DNA which has been introduced. Preferably, the selectable marker gene is one which facilitates selection at the tissue culture stage of the trait-associated gene identification methods of the invention, e.g., a kanamyacin, hygromycin or ampicillin resistance gene.

The selection of an appropriate promoter effective to express the selectable marker-encoding sequence and the termination element for the selectable marker-encoding sequence may be accomplished by the use of well known, and/or commercially available sequences.

An exemplary vector for use in the methods of the invention is the pSKI15 plasmid. (See, http://www.biosun.asalk.edu/LABS/pbio-w/index.html; Hayashi et al., Science 258: 1350-1353, 1992; Walden et al., Plant Mol Biol 26:1521-1528)

The key elements of pSKI15 are; (a) a pBstKS+segment from the Bluescript™ plasmid, with an E. coli origin of replication (Stratagene), (b) the backbone from the RK2 plasmid, located between the left and right borders of the T-DNA, which contains the oriV and oriT regions responsible for stable replication in Agrobacterium; (c) a bialaphos resistance (BAR) gene encoding a phosphinothricin acetyltransferase enzyme; (d) a mannopine synthase (mas) promoter operatively linked to BAR gene, upstream thereof; (e) an octapine synthase (ocs) polyA termination element located downstream of the BAR gene, adjacent the left border of the plasmid, and (f) a tandem duplicated 35S enhancer element (3X).

Generally, the construction of vectors for use in practicing the present invention are known by those of skill in the art. (See generally, Maniatis, et al., MOLECULAR CLONING: A LABORATORY MANUAL, 2d Edition (1989), and Ausubel, F. M., et al., Eds., CURRENT PROTOCOLS IN MOLECULAR BIOLOGY, John Wiley & Sons, Inc., Copyright (c)1987, 1988, 1989, 1990, 1993 by Current Protocols; Gelvin, S. B., Schilperoort, R. A., Varma, D. P. S., eds. Plant Molecular Biology Manual (1990), all three of which are expressly incorporated by reference, herein.

Preferred vectors for use in the trait-associated gene identification methods of the invention supply the left and right border sequences of Agrobacterium for insertion into the host genome and an enhancer sequence which facilitates enhanced expression of native plant genes by acting on endogenous promoters.

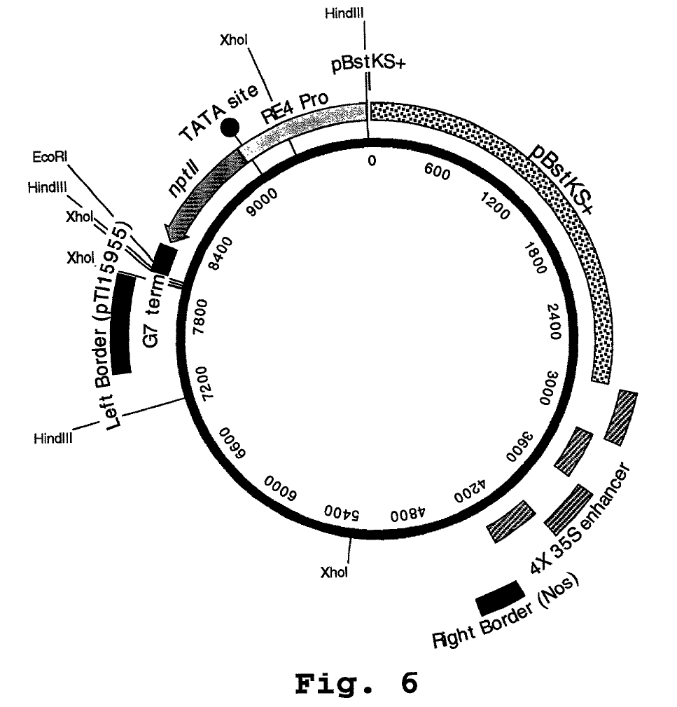

Exemplary transformations are carried out using colonies of Agrobacterium tumefaciens strains EHA 105, EHA 101 or GV3101 containing a binary plasmid, e.g., pAG3201(pSKI backbone with a 4x duplicated 35S enhancer and the nptII gene under the control of a CsVMV promoter), pAG3202 (pSKI backbone with a 4x duplicated 35S enhancer and the nptII gene under the control of an RE4 promoter, FIG. 6) or pAG4201 (pPZP-200 backbone with a 4x duplicated 35S enhancer and the nptII gene under the control of an RE4 promoter).

Enhanced transcription in plants may find use in enhancing the expression of endogenous (native) or modified endogenous (i.e., non-native) genes.

IV. Methods Of Transforming Plants

Plants for use in carrying out the trait-associated gene identification methods of the invention must have the following properties; (1) the ability to be infected with Agrobacterium sp., (2) the ability to be grown in large numbers in a short time frame, (3) the ability to produce a fleshy fruit and (4) traits or phenotypes, which are observable or easily evaluated.

The methods of the invention are directed to fruit-bearing plants. However, it will be understood that the use of the methods described herein are not limited to any particular fruit-bearing plant, and are generally applicable to plants which produce fleshy fruits; for example, Lycopersicum (tomato), Vitas (grape), Fragaria (strawberry), Rubus (raspberry, blackberry, loganberry), Ribes (currants and gooseberry), Vaccinium, (blueberry, bilberry, whortleberry, cranberry), Actinida (kiwifruit and Chinese gooseberry), drupe fruits; including, but not limited to, Malus (apple) and Pyrus (pear), Cucumis sp. (melons), most members of the Prunus genera, sapota, mango, avocado, apricot, peaches, cherries, plums, and nectarines.

Dwarf varieties of fruit-bearing plants are preferred for practicing the methods of the invention. In particular, dwarf varieties of tomato, including but not limited to Micro-Tom, Florida Petite, Tiny Tim and Small Fry are preferred.

Dwarf tomatoes are characterized by their short internodes which give plants a compact appearance. The miniature Lycopersicon esculentum cultivar, Micro-Tom (Micro tomato) is a proportionally dwarfed plant which grows at high density (up to 1357 plants/m⁻²), has a short life cycle (70-80 days from sowing to fruit ripening), and for which fruit size, and leaf size have been genetically reduced. (Meissner et al., The Plant Journal 12(6) 1465-1472, 1997; Scott, J. W. and Harbaugh, B. K., University of Fla. Circular S-370, Dec. 1989) In addition, Micro-Tom has been shown to be resistant to a number of diseases and can be transformed at frequencies of up to 80% through Agrobacterium-mediated transformation of cotyledons (Meissner et al., 1997).

Similar to Micro-Tom, Florida Petite (Fla. Agr. Expt. Sta. Circ. S-285), Tiny Tim and Small Fry are dwarf varieties of tomato which have a short life cycle, and for which fruit size, and leaf size have been genetically reduced.

The optimal procedure for transformation of plants with Agrobacterium vectors, will vary with the type of plant being transformed. Exemplary methods for Agrobacterium-mediated transformation include transformation of explants of hypocotyl, shoot tip, stem or leaf tissue, derived from sterile seedlings and/or plantlets. Such transformed plants may be reproduced sexually, or by cell or tissue culture.

Generally, transformation of tomato (L. esculentum) has been accomplished using injured cotyledon tissue, particularly cotyledon tissue co-cultured with Agrobacterium tumefaciens and feeder cells (also termed “nurse cultures”). (See, U.S. Pat. No. 5,565,347; Fillati J., et al., Biotechnology 5: 726-730, 1987) Explants such as cotyledon tissue, which are not derived in vitro must be surface sterilized prior to use, which can damage the cells and thereby interfere with the regeneration potential of the tissue.

Leaf disc transformation of tomato using Agrobacterium tumefaciens has also been reported (McCormick, et al., Plant Cell Reports 5:81-84, 1986).

Methods for transformation of in vitro grown explants, such as hypocotyl tissue provide advantages over other methods for transformation of tomato, in that the tissue is uniform and sterile and need not be wounded or surface sterilized prior to infection, in contrast to growth chamber or greenhouse grown plants.

Hypocotyl transformation has been described for Brassica sp. in U.S. Pat. Nos. 5,750,871 and 5,463,174, which are directed to methods involving tobacco feeder cells, which act as a nurse culture for the Brassica explant.

A method for transformation of hypocotyl explants from tomato, which relies on the use of feeder cells or nurse cultures is described in Frary A., and Earle E. D., Plant Cell Reports 16: 235-240, 1996.

In one preferred embodiment of the invention, an improved hypocotyl transformation method which generally does not require the use of feeder cells or nurse cultures is employed to introduce Agrobacterium vectors into plant cells (see Example 1).

Also preferred, is the introduction of Agrobacterium vectors into plant cells by shoot tip transformation. A preferred method for shoot tip transformation does not require feeder cells or nurse cultures, and is also presented in Example 1.

In a further preferred embodiment of the invention, floral tissues are dipped into a solution containing Agrobacterium tumefaciens, 5% sucrose and a surfactant Silwet L-77, as described in Cough, S. J. and Bent, A. F., the Plant Journal 16(6): 735-743 (1998).

Transformed explant cells are screened for the ability to be cultured in selective media having a threshold concentration of selective agent. Explants that can grow on the selective media are typically transferred to a fresh supply of the same media and cultured again. The explants are then cultured under regeneration conditions to produce regenerated plant shoots. After shoots form, the shoots are transferred to a selective rooting medium to provide a complete plantlet. The plantlet may then be grown to provide seed, cuttings, or the like for propagating the transformed plants. The method provides for high efficiency transformation of plant cells with enhanced expression of native plant genes and regeneration of plants having modified traits or phenotypes associated with enhanced expression of particular native plant genes.

V. Detecting And Characterizing Enhanced Gene Expression

Transformed plant cells grown under selective conditions will yield mature plants which are screened for desired traits. It will be appreciated that seeds derived from such transformed plants may be germinated and grown to yield mature plants that may also be screened for desired traits.

Of particular interest are biochemical modifications of plants and fruits which result in a change in the level of vitamins, minerals, elements, amino acids, carbohydrates, lipids, nitrogenous bases, isoprenoids, phenylpropanoids or alkaloids.

Plant output traits of interest include resistance to fungal, bacterial and viral pathogens, plant insect resistance; modified flower size, modified flower number, modified flower pigmentation and shape, modified leaf number, modified leaf pigmentation and shape, modified seed number, modified pattern or distribution of leaves and flowers, modified stem length between nodes, modified root mass and root development characteristics, and increased drought, salt and antibiotic tolerance.

Fruit-specific output traits of interest include modified lycopene content, modified content of metabolites derived from lycopene including carotenes, anthocyanins and xanthophylls, modified vitamin A content, modified vitamin C content, modified vitamin E content, modified fruit pigmentation and shape, modified fruit ripening characteristics, fruit resistance to fungal, bacterial and viral pathogens, fruit resistance to insects, modified fruit size, and modified fruit texture, e.g., soluble solids, total solids, and cell wall components.

The invention further includes plant metabolites (chemicals) which are produced by the plant in response to enhanced expression of one or more native plant genes, resulting in a phenotype or trait of interest. It will be understood that once identified and characterized, such chemicals may be produced by the plant using recombinant DNA techniques or produced synthetically using standard techniques for chemical synthesis known to those of skill in the art.

A. Identification Of Genes Associated With The Desired Phenotype The genes associated with a particular phenotype or “trait” of interest are identified by proximity to the Ti tagging construct. Genes that are tagged by T-DNA insertions can be cloned and sequences in the host genome that flank the T-DNA sequence used as probes in the cloning of wild-type genes. These techniques have been described by Feldman et al., Science 243: 1351-1354, 1989; Marks and Feldman, Plant Cell 1:1053-1050, 1989; and Hayashi et al., Science 258:1350-1352, 1992).

The genes of interest are identified and DNA sequences of interest isolated by plasmid rescue and/or conventional genome walking techniques. Plasmid rescue is further described in Behringer and Medford, Plant Mol. Biol. Rep. 10(2):190-198, 1992. Reagents for genome walking are commercially available (e.g., GenomeWalker™ from Clontech, Palo Alto, Calif.).

As described above, the role of the identified gene(s) in the selected phenotype is confirmed by preparing transgenic plants with a separate conventional plant gene-expression vector for each identified gene.

VI. Evaluation Of Transformation

Following introduction of Agrobacterium vectors into plant cells using the trait-associated gene identification methods of the invention, the transformation of plant tissue and analysis of genes and gene products associated with traits of interest can be confirmed by a variety of methods. Exemplary methods include analysis of nucleic acids, proteins, and metabolites associated with the expressed gene, as described below.

A. PCR Analysis

DNA is extracted from various plant tissues and analyzed for the presence of a gene of interest by polymerase chain reaction (PCR) procedures routinely employed by those of skill in the art. PCR is carried out using oligonucleotide primers specific to Agrobacterium vector sequences adjacent the gene of interest or specific to the gene itself, once the sequence has been determined. (See, e.g., Jensen, L. G., et al., Proc. Natl. Acad. Sci. USA 93:3487-3491, 1996.)

B. RT PCR Analysis

RNA may also be extracted from various plant tissues, followed by reverse transcription of mRNA and amplification of partial cDNA sequences using polymerase chain reaction (PCR).

C. Southern Analysis

Transformation of each plant can be confirmed using Southern blot analysis of genomic DNA. Typically, total DNA is isolated from each transformant (e.g., Schwarz-Sommer, et al., 1984). The DNA is then digested with restriction enzyme, fractionated in an agarose gel and transferred to nitrocellulose filters (e.g., HYBOND-N, Amersham) according to standard techniques. The blot is then probed, e.g., with ³²P-labeled target cDNA. Procedures for restriction digestion, gel electrophoresis, Southern transfer and hybridization are as described by Maniatis et al., 1989, expressly incorporated by reference herein.

D. Northern Analysis

RNA is isolated from specific plant tissues, separated, e.g., in a 1.2% agarose gel containing 2.2M formaldehyde, and blotted to a nylon filter, e.g., Hybond-N, according to the standard procedures routinely used in the art. Strand specific RNA probes are synthesized by phage T7 and T3 RNA polymerases from a cDNA clone associated with the desired trait and hybridized to the RNA on the filter. This allows for a determination of the presence and an estimation of the amount of mRNA resulting from expression the target gene. Northern analysis of rescued plant RNA may be used to look for overexpression and characterize the expressed gene, e.g. tissue or developmental stage specific expression. Procedures for Northern analysis are as described by Maniatis et al., 1989.

E. Western Blots and Immunoassays, and the Like

Western blot analysis may be conducted on putative transformants to detect the presence of a protein encoded by a gene using standard techniques for Western blotting such as the protocol described in Glick, B. R. and Thompson, J. E., Eds. METHODS IN PLANT MOLECULAR BIOLOGY AND BIOTECHNOLOGY, p213-221, CRC Press, 1993.

VII. Modes Of Practicing The Invention

An important aspect of the Agrobacterium vectors for use in the trait-associated gene identification methods of the invention is that the 35S enhancer components of the vectors operate to enhance the transcription of native plant genes which are located within 5000 or more bp of the enhancer insertion site; however, the native plant genes need not be linked to the CaMV 35S enhancer sequence directly through a gene promoter.

A preferred aspect of the methods of the invention is effective transformation of a large number of plant cells by the hypocotyl or shoot tip transformation methods, as described herein.

A further preferred aspect of the invention is the transformation of plants having short life cycles as exemplified by dwarf varieties of tomato, such that the identification of genes associated with traits of interest may be accomplished in a short period of time.

VIII. Applications Of The Method

From the foregoing, it can be appreciated that the methods of the present invention offer broad applicability to situations wherein it is desirable to develop fruit-bearing plants having modified traits.

It will be understood that any of the methods described herein are readily adaptable to a kit format for use in routine screening of plants for modified traits associated with enhanced expression of native plant genes.

All patent and literature references cited in the present specification are hereby incorporated by reference in their entirety.

While the invention has been described with reference to specific methods and embodiments, it will be appreciated that various modifications and changes may be made without departing from the invention.

Materials and Methods.

Micro-Tom Tomato Genomic DNA Extraction

Nucleon™ PhytoPure™ systems from Amersham™ was used for extracting genomic DNA using Nucleon Phytopure, Plant and fungal DNA extraction kits.

1.0 g of fresh plant tissue was ground in liquid nitrogen to yield a free flowing powder, then transferred to a 15 ml polypropylene centrifuge tube. 4.6 ml of Reagent 1 from the Nucleon Phytopure kit was added with thorough mixing, followed by addition of 1.5 ml of Reagent 2 from the Nucleon Phytopure kit, with inversion until a homogeneous mixture is obtained. The mixture is incubated at 65° C. in a shaking water bath for 10 minutes, and placed on ice for 20 minutes. The samples are removed from the ice, 2 ml of −20° C. chloroform added, mixed and centrifuged at 1300 g for 10 minutes. The supernatant is transferred into a fresh tube, 2 ml cold chloroform, 200 μl of Nucleon PhytoPure DNA extraction resin suspension added and the mixture shaken on a tilt shaker for 10 minutes at room temperature, then centrifuged at 1300 g for 10 minutes. Without disturbing the Nucleon resin suspension layer, the upper DNA containing phase is transferred into a fresh tube, centrifuged at 9500 rpm for 30 minutes to clarify the transferred aqueous phase if the upper phase appears cloudy, an equal volume of cold isopropanol added, and the tube is gently invert the tube until DNA precipitates and then it is pelleted by centrifugation, then washed with cold 70% ethanol, pelleted and air-dried.

DNA is resuspended in TE buffer (10 mM Tris. HC1, pH 7.4, 1 mM EDTA), containing RNase, incubated at 55° C. for 15 minutes, further extracted phenol/chloroform, then chloroform, run on a 1% agarose gel to check the DNA Quality, the DNA concentration determined by a DNA fluorometer (Hoeffer DyNA Quant 200).

Plasmid Rescue

Genomic DNA from single copy T-DNA insertion lines identified by Southern hybridization is digested by the restriction enzymes used in Southern Hybridization. The restriction fragments are then self-ligated and used to transform the E. coli cells. The plasmids that contain a full-length pBluescript vector, 4X 35S enhancer, and a right border T-DNA flanking genomic DNA fragment are rescued.

Genomic DNA is digested with a selected restriction enzyme under standard reaction conditions. Briefly, the restriction enzyme is heat inactivated at 65° C. for 20 minutes, phenol/ chloroform and chloroform isoamyl (24:1) extracted once with each, then put into a ligation reaction containing the following:

| | |
|---|---|
| Digested Genomic DNA | 40 μl |
| 5X Ligation Buffer | 50 μl |
| Ligase (Gibcol, 1 U/μl) | 10 μl |
| ddH₂O | 150 μl |

The ligation reactions are left at 16° C. overnight, the ligated DNA is precipitated, resuspended in ddH20 and used to transform E. coli SURE cells (Stratagene) via electroporation, with 10 pg of pUC18 plasmid as a control.

The transformation mixture is spread on two LB-plates containing 100 μg/ml ampicillin and incubated overnight at 37° C. Single colonies are picked from the pates and used to start a 5 ml LB-ampicillin broth culture of each overnight at 37° C. The plasmid is extracted from the culture and restriction digested to confirm the size of genomic insertion.

Sequencing Of Rescued Plasmids

Sequencing is accomplished using a ABI Prism BigDye™ Terminator Cycle Sequencing Ready Reaction Kit (PE Applied Biosystem), AmpliTaq DNA Polymerase (Perkin-Elmer), an ABI Prism™ 310 Genetic Analyzer (Perkin-Elmer) and sequence analysis software, e.g., Sequencer™ 3.1.1 or MacVector 6.5.3.

Primers for sequencing are designed based on the sequence of the left end of plasmids, as exemplified by a sequence from (a) inside the Hind III or Kpn I site, e.g., an M13 reverse primer (SEQ ID NO: 9); (b) inside the Xho I site, e.g., SEQ ID NO: 10; or (c) inside the Eco RI site, e.g., SEQ ID NO :11.

An ABI Prism BigDye™ Terminator Cycle Sequencing Ready Reaction Kit is used to sequence a plasmid using a ABI Prism™ 310 Genetic Analyzer following the protocols from manufacture.

The identified genomic insertion sequence is used to do NCBI BLAST™ similarity search using the interface provided at “http://www.ncbi.nlm.nih.gov/BLAST/”. The BLAST search results indicate the presence or absence of related sequences which have been deposited in the public databases that are searched, as of the date of the search.

In general, the largest rescued plasmid is used to design new primers to sequence the full-length genomic insertion. Such primers may be designed using a computer program, for example, the Primer3 program found at “http://www.genome.wi.mit.edu//cgi-bin/primre/primer 3 www.cgi/”.

Restriction deletion of the rescued plasmid may be applied to speed up sequencing in this procedure. This is accomplished by preparing a restriction digest of the rescued plasmid and self ligation of the digested plasmid using different restriction enzymes, such as BamHI, Spe I, Bst XI and Eco RV that cut right border of T-DNA including the 4X 35S enhancer. A restriction digest of the self-ligated plasmid is used to confirm the deletion of the right border of T-DNA and genomic insertion, then the deleted right border of the genomic insertion is sequenced using a T7 primer.

The presence of open reading frames is then determined using a computer program, as exemplified by the GENSCAN Web Server at MIT: http://CCR-081.mit.edu/GENESCAN.html

When no open reading frame is predicted based on the sequence of the rescued genomic fragment, a BAC library may be screened to clone a large fragment flanking the right and left border of the T-DNA. In carrying out such as screen, a rescued genomic fragment is used as a probe to screen a high density membrane (Research Genetics, Inc.) spotted with a whole Tomato BAC library (RGLEMOG1, Research Genetics) in order to identify positive clones that contain large T-DNA flanking sequences for determining the activated genes in mutant plants.

When an open reading frame is predicted based on the sequence of the rescued genomic fragment, RT-PCR and/or Northern blots are used to correlate the presence of the identified open reading frame with RNA isolated from plants having the observed phenotype.

Once confirmed, the identified gene (coding sequence) is re-introduced back into a wild type plant to confirm gene function.

The following examples illustrate, but in no way are intended to limit the present invention.

### EXAMPLE 1

Generation Of Mutants Using Activation Tagging In Micro-Tom Tomato

Activation tagging mutants were generated in tomato cv. Micro-Tom using Agrobacterium-mediated transformation. Sterile seedlings and plantlets were used as the source of explants. More specifically, hypocotyl, shoot tip, stem and leaf were transformed.

Seeds of (Lycopersium esculentum) were surface sterilized in 25% bleach with tween-20 for 15 minutes and rinsed with sterile water before plating on seed germination medium (MS salts, Nitsch vitamins, 3% sucrose and 0.7% agar, pH 5.8). The basic germination medium may be modified by the addition of auxin and/or cytokinins and giberrellic acid as necessary. The cultures were incubated at 24° C. with a 16 hr photo period (50-60 μmol.m⁻²s⁻¹). Seven to ten day old seedlings and one month old in vitro plants were used for hypocotyl/ shoot tip and stem/leaf explants respectively.

Single colonies of Agrobacterium tumefaciens strains EHA 105/EHA 101/GV3101 containing the binary plasmid pAG3202 (FIG. 6) were grown in MGL medium at pH 5.4 overnight and diluted to 5×10⁸ cells/ml with MGL or liquid plant co-cultivation medium.

Hypocotyls and stems were cut into 3-5 mm segments, then immersed in bacterial suspension, blotted on sterile filter paper and placed on co-cultivation medium. In vitro raised seedlings, 7-8 day old, were used as the source of shoot tip explants. Shoot-tips 3-6 mm, were longitudinally segmented using a dissecting scope in a petri plate in the presence of the Agrobacterium suspension. In the case of leaf explants, the mid portion was cut into 2-4 mm cross sections after removing the petiole end and the leaf tip. The youngest two or three leaves are preferred as the older leaves tend to be less morphogenetic in culture.

In each case, the explants were immersed in bacterial suspension, blotted on sterile filter paper and placed on co-cultivation medium (MS salts, LS vitamins, 3% sucrose, 0.1 mg/l kinetin, 0.2 mg/l 2,4-D, 200 mg/l potassium acid phosphate, 50 μM acetosyringone and 0.7% agar, pH 5.4) for 2-3 days.

After two to three days of co-culture, the explants were transferred to shoot regeneration medium containing MS salts, Nitsch vitamins, 3% sucrose, 2 mg/l zeatin, 500 mg/l carbenicillin, 200 mg/L timetin and 0.7% agar at pH 5.8, supplemented with the antibiotic, kanamycin at 75 - 400 mg/l depending on the promoter for nptII. The selection level of antibiotic was gradually raised over an 8 weeks period based on the tissue response.

The explants were transferred to fresh medium every two weeks. Initiation of callus with signs of shoot initials was observed from 3-6 weeks depending on the type of explant. Callusing and shoot regeneration has been observed to continue over approximately 4 months after which the explant tissues decline. A mixture of green and bleached shoots were observed among the regenerants. Green shoots of approximately 1 cm in size with distinct shoot meristems were excised from the callus and transferred to root induction medium containing MS salts, Nitsch vitamins, 3% sucrose, 1 mg/l IBA, 50 mg/l kanamycin, 100 mg/l carbenicillin or 100 mg/L timetin and 0.7% agar, pH 5.8. The rooted plants were out-planted to soil in a Biosafety greenhouse.

The frequency of transformation was calculated as the number of rooted plants in presence of selection (kanamycin) relative to the total number of explants, expressed as a percentage. The average observed transformation frequency was in the range of 8 to 54%.

Plants were transported to greenhouse facilities, potted up in 3.5″ pots tagged for plant identification.

After the plants were established in the greenhouse, they were observed for phenotypic variations relative to wild-type Micro-Tom plants. To achieve this, several wild-type plants are kept in close proximity to the transgenic plants. Each plant is observed closely twice a week with observations noted and documented by photographs.

Observation of the morphological characteristics of approximately 2000 plants generated by activation tagging has indicted the presence of a number of interesting morphological mutants, with exemplary phenotypes summarized in Table 1.

TABLE 1
Micro-Tom Tomato Activation Tagging Mutants.

| Identifier | Observed phenotype |
|---|---|
| H000000012 | Upward-curling leaf margins |
| H000000013 | Leaves larger than wild-type; smooth margins |
| H000000028 | Larger leaflets |
| H000000046 | Darker leaf color |
| H000000056 | Upward-curling leaf margins; unusual leaf shape |
| H000000098 | Unusual leaf shape; upward-curling leaf margins; some |
| | simple leaves |
| H000000151 | Large leaflets |
| H000000152 | long internodes |
| H000000154 | Rounded leaf shape; leaflets overlapping |
| H000000164 | only 3 leaflets on leaves instead of 5-7; dwarf |
| L000000015 | Dwarf |
| L000000023 | Unusual floral organs; macro-calyx, petals pale green, |
| | anthers separate and not fused like in wild, anthers persist- |
| | ent after fruit set, possible sterility; lobed fruits, split |
| | fruits, fruits with protruding anthers |
| T000000003 | pale light green leaves |
| T000000004 | Dwarf |

### Example 2

Exemplary Mutant With Delayed Flowering Phenotype

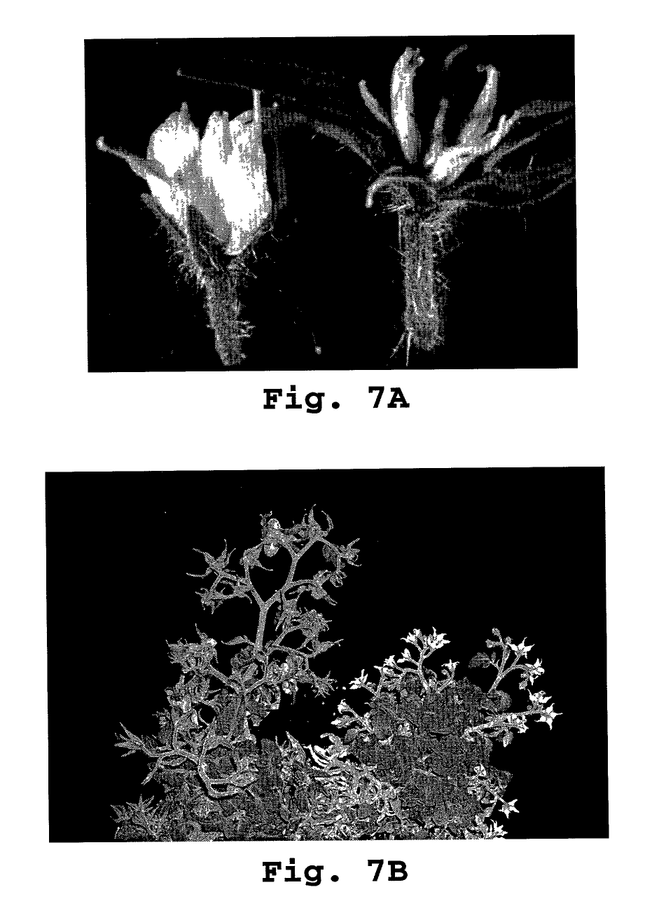

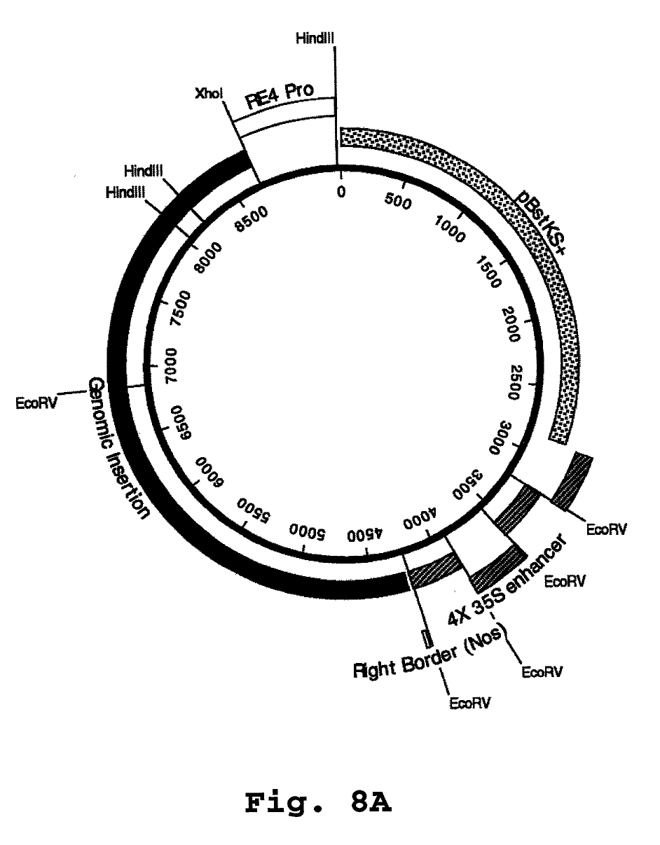

An exemplary activation tagged mutant, identified as “L000000023”, derived from leaf explants and generated as described in Example 1, was designated “L23”. (See FIGS. 7A and B).

Micro-Tom genomic DNA was extracted in sufficient yield and quality for plasmid rescue of activation tagged plant lines using the Nucleon™ PhytoPure™ system from Amersham™ following the protocol suggested by the manufacturer, except that the resuspended DNA was further extracted with phenol/chloroform.

The floral mutant (L23) was identified from less than 200 individual Micro-Tom tomato ACTTAG lines that were transformed by the binary plasmid (pAG-3202) which contains four copies of the 35S enhancer and a full-length pBstKS+ vector in T-DNA (FIG. 6).

PCR Characterization Of Micro-Tom Tomato.

Primers specific to the pBluescript and 35S enhancer region of pAG3202 were used to characterize control and T1 or T2 activation tagged plant lines by PCR.

PCR was carried out using AmpliTaq DNA Polymerase Kits, AmpliTaq DNA, 10X PCR buffer, 25 mM MgCl2 and dNTP (10 mM, Perkin Elmer), genomic DNA at 15 ng/μl and a DNA Thermal Cycler 48 (Perkin Elmer Cetus).

The following primer sequences were used:

35S enhancer primers

| | |
|---|---|
| 35S 5′ forward (GAT CCC CAA CAT GGT GGA G) | (SEQ ID NO: 12) |
| 35S 3′ reverse (CAC ATC AAT CCA CTT GCT TTG) | (SEQ ID NO: 13) |

pBluescript primers

| | |
|---|---|
| pBKS + forward, (ACT ACG ATA CGG GAG GGC TT) | (SEQ ID NO: 14) |
| pBKS + reverse, (CTG GCG TAA TAG CGA AGA GG) | (SEQ ID NO: 15) |

A typical thermal cycling program was carried out. For example:

94° C. for 2 minutes; 30 cycles of 94° C. for 30 seconds, 57° C. for 1 min and 72° C. for 1 min; followed by 72° C. for 7 minutes and an unlimited time at 4° C.

Following PCR, the products were separated on 1% agarose gels by electrophoresis and visualized by staining with Ethidium Bromide.

PCR was carried out using p3202, wild type Micro-Tom DNA, L23 Micro-Tom DNA and a control which lacked DNA together with primers specific to either the 35S enhancer or pBluescript KS. The results showed that three fragments were amplified from both pAG-3202 plasmid and the mutant DNA with a size about 300 bp, 600 bp and 900 bp respectively using the 35S primers, and a 1.5 kb fragment was amplified in pAG-3202 plasmid and the mutant DNA using pBstKS+ primer. The results of PCR with primers specific to the 35S enhancer indicated the presence of the 35S enhancer sequence in p3202 and L23 Micro-Tom DNA, but not wild type Micro-Tom DNA or the negative control without DNA. The results of PCR with primers specific to pBluescript KS, indicated the presence of the pBluescript KS sequence in the pAG3202 plasmid DNA and L23 Micro-Tom DNA, but not wild type Micro-Tom DNA or the negative control without DNA, suggesting that genomic integration of the activation tagging DNA had occurred in the L23 mutant, and neither the 35S enhancer sequence nor pAG3202 plasmid DNA was present in wild type Micro-Tom or the negative control.

Genomic DNA of ACTTAG lines was digested by certain restriction enzymes that only cut pAG-3202 T-DNA left border and separated by gel electrophoresis. The fractionated DNA was then transferred to a nylon membrane and probed with ³²P labeled pBstKS+ fragment to probe the Eco RI, Hind III and Xho I digested wild type and mutant genomic DNA to determine the number of T-DNA insertions in each activation tagged line.

Results indicated the presence of one hybridization band on Southern blots from each restriction digestion of DNA derived from L23 mutant plants and no hybridization signal on Southern blots of DNA derived from wild type plants, indicating that L23 is a single T-DNA insertion line.

Genomic DNA from the L23 was plasmid rescued according to the protocol detailed above, and the flanking sequences analyzed.

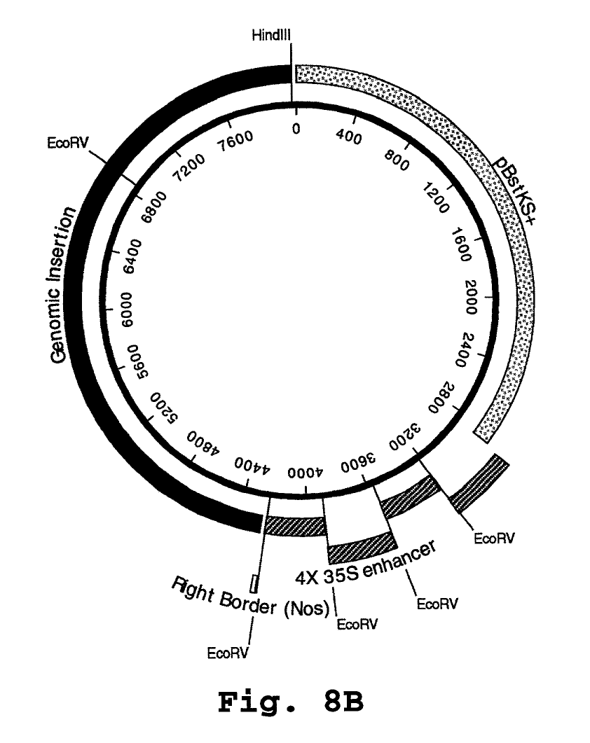

Two different sized plasmids with a 3.7 kb and 4.5 kb genomic insertion fragment were rescued from Hind III and Xho I digestion of L23 genomic DNA respectively (FIGS. 8A and 8B). The large fragment of the rescued genomic insertion was sequenced using ABI Prism™ 310 Genetic Analyzer (Perkin-Elmer) according to the protocol detailed above. In addition, a BstXI partial digestion of an XhoI rescued plasmid was preformed, self-ligated and sequenced with a T7 primer (SEQ ID NO: 22) designed to amplify the right border of pBstKst.

The primers used for sequencing are presented as SEQ ID NO: 9 and SEQ ID NO: 16-SEQ ID NO: 25.

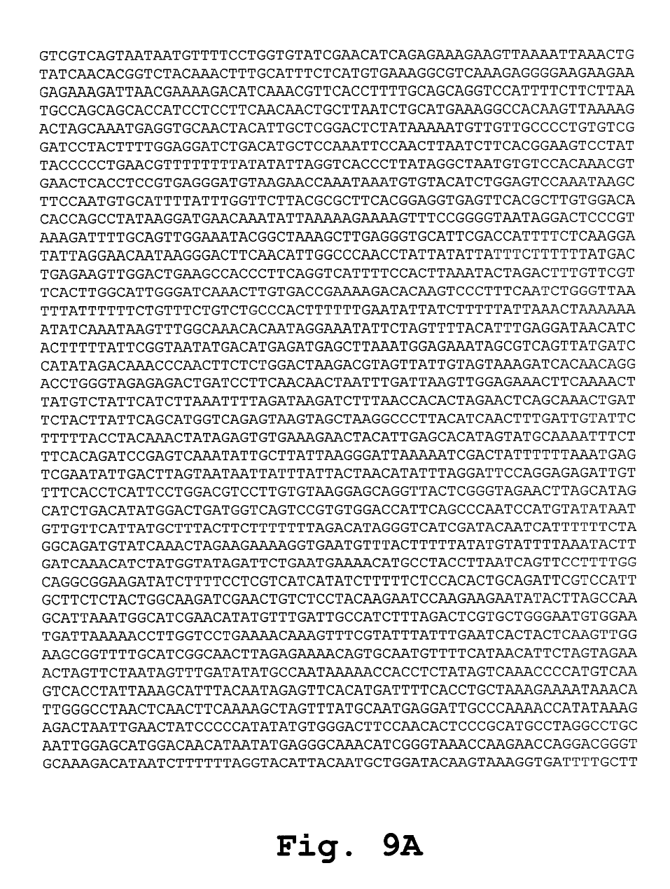

The sequencing resulted in identification of a 4437 bp DNA sequence (FIGS. 9A-9B, SEQ ID NO: 26). A Basic BLASTN search (http://www.ncbi.nlm.nih.gov/BLAST) of non-redundant nucleic acid sequence databases, conducted on Feb. 29, 2000, through NCBI (http://www.ncbi.nlm.nih.gov/index.html) with the nucleotide sequence presented in FIGS. 9A-9B revealed no significant sequence identity between sequences available in GenBank and nucleic acids 1-4437 of the SEQ ID NO :26.

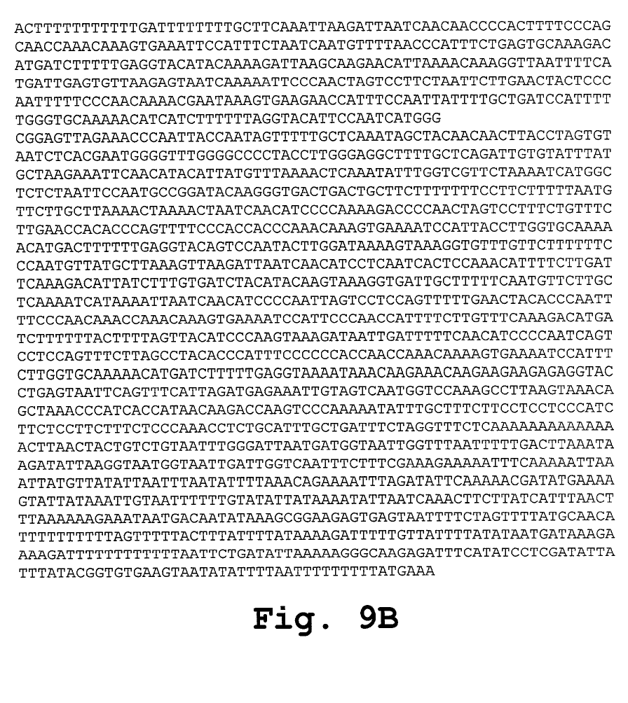

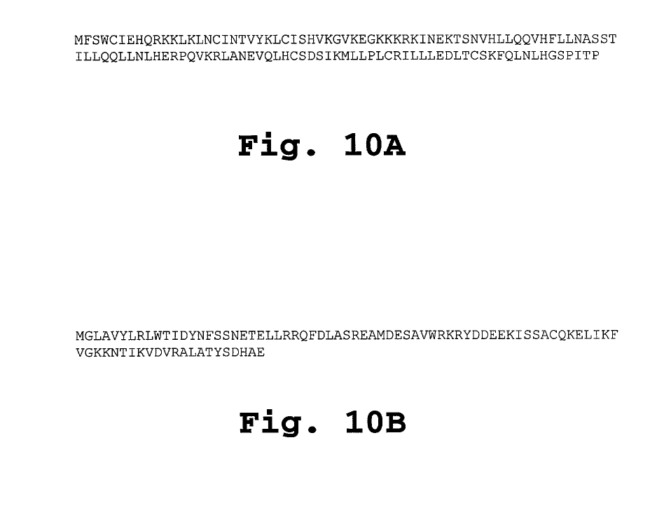

Two open reading frames were predicted in the rescued sequence using the GENESCAN computer program found at “MIT http://CCR-081.mit.edu/GENESCAN”, indicating the presence of genes which encode polypeptides of about 124 and 85 amino acids, respectively (FIG. 10A, SEQ ID NO: 27 and FIG. 10B, SEQ ID NO: 28, respectively).

It is claimed:

1. A method for identifying genes associated with a desired trait in a fruit-bearing plant comprising:
(i) transforming cells of a plant with a plant cell expression vector having an E. coli origin of replication, an element which functions to enhance gene expression, a selectable marker-encoding nucleotide sequence operably linked to a promoter effective to express the selectable marker encoding sequence, a termination element for said selectable marker-encoding nucleotide sequence, and a T-DNA sequence;
(ii) in a manner effective to express said selectable marker-encoding nucleotide sequence;
(ii) selecting plant cells which have been transformed by their ability to grow in the presence of an amount of selective agent that is toxic to non-transformed plant cells;
(iii) regenerating transformed plant cells to yield mature plants;
(iv) selecting plants having a desired trait; and
(v) identifying, isolating and characterizing genes the transcription of which was enhanced by said element which functions to enhance gene expression.

2. The method of claim 1, further comprising the steps of
(vi) preparing a separate heterologous gene construct for each isolated gene;
(vii) transforming plants with said separate heterologous gene construct in a manner effective to enhance expression of the isolated gene in said plants;
(vii) selecting plants having the desired trait.

3. The method of claim 1, wherein the element which functions to enhance gene expression is selected from the group consisting of a CaMV 35S enhancer element, a Figwort Mosaic Virus (FMV) promoter sequence, a peanut chlorotic streak caulimovirus full-length transcript (PClSVFLt) sequence and a mirabilis mosaic virus (MMV), promoter sequence (SEQ ID NO: 8).

4. The method of claim 2, wherein said CaMV 35S enhancer element is a 4X tandem duplicated CaMV 35S enhancer element having the sequence presented as SEQ ID NO: 1.

5. The method of claim 2, wherein said Figwort Mosaic Virus (FMV) sequence is the promoter sequence presented as SEQ ID NO: 5 or the enhancer sequence presented as SEQ ID NO: 6.

6. The method of claim 2, wherein said peanut chlorotic streak caulimovirus full-length transcript (PClSVFLt) sequence is the enhancer sequence presented as SEQ ID NO: 7.

7. The method of claim 2, wherein said mirabilis mosaic virus (MMV) sequence is the promoter sequence presented as SEQ ID NO: 8.

8. The method of claim 1, wherein said selectable marker is a polypeptide which confers herbicide-resistance to transformed plant cells expressing said marker.

9. The method of claim 1, wherein said selectable marker is an antibiotic resistance gene which confers resistance to an antibiotic selected from the group consisting of kanamycin, G418, bleomycin, hygromycin, chloramphenicol, ampicillin and tetracycline.

10. The method of claim 8, wherein said antibiotic is kanamycin.

11. The method of claim 1, wherein said fruit-bearing plant is a dwarf plant.

12. The method of claim 10, wherein said dwarf plant is a tomato plant.

13. The method of claim 1, wherein said transforming cells includes Agrobacterium tumifaciens-mediated transformation.

14. The method of claim 12, wherein said transforming cells includes introduction of Agrobacterium tumifaciens into hypocotyl tissue derived from said fruit-bearing plant.

15. The method of claim 12, wherein said transforming cells includes introduction of Agrobacterium tumifaciens into shoot tip tissue derived from said fruit-bearing plant.

16. The method of claim 1, wherein said desired trait is a biochemical modification of a plant and fruit selected from the group consisting of a change in the level of vitamins, a change in the level of minerals, or elements, a change in the level of amino acids, a change in the level of carbohydrates, a change in the level of lipids, a change in the level of nitrogenous bases, a change in the level of isoprenoids, a change in the level of phenylpropanoids and a change in the level of alkaloids.

17. The method of claim 1, wherein said desired trait is a fruit-bearing plant specific trait selected from the group consisting of increased resistance to fungal, bacterial or viral pathogens, increased resistance to insects, modified flower size, modified flower number, modified flower pigmentation and shape, modified leaf number, modified leaf pigmentation and shape, modified seed number, a modified pattern or distribution of leaves and flowers, modified stem length between nodes, modified root mass or root development characteristics, and increased drought, salt and antibiotic tolerance.

18. The method of claim 1, wherein said plant cell expression vector is selected from the group consisting of pSKI15, pAG 3201, pAG 3202 and pAG 4201.

19. A transgenic fruit-bearing plant comprising enhanced expression of a gene identified by the method of claim claim 1, 16 or 17.
